(19) United States
(12) Patent Application Publication
(10) Pub. No.: US 2017/0071992 A1
(43) Pub. Date: Mar. 16, 2017

(54) METHOD AND SYSTEM FOR EXTRACTING COMPOUNDS FROM PLANTS AND PLANT BASED MATERIALS

(71) Applicant: MRX XTRACTORS LLC, Tigard, OR (US)

(72) Inventor: Paul TOMASO, Tigard, OR (US)

(21) Appl. No.: 15/264,833

(22) Filed: Sep. 14, 2016

Related U.S. Application Data
(60) Provisional application No. 62/218,664, filed on Sep. 15, 2015.

(51) Int. Cl. A61K 36/185; A61K 31/56; B01D 11/02; A61K 31/352

(52) U.S. Cl. CPC A61K 36/185; A61K 31/352; A61K 31/56; B01D 11/0203; B01D 11/0288; A61K 2236/37

(57) ABSTRACT

The invention provides a method and system for extracting desired compounds from a plant or a plant based material featuring a) providing a plant or plant based material; b) pumping CO₂ gas from a CO₂ storage vessel into an apparatus containing a CO₂ circulation loop; c) circulating CO₂ through the plant or plant based material in the circulation loop; d) extracting the desired compounds; and e) discharging the CO₂ gas from the CO₂ circulation loop. The circulation loop may have a separator vessel and an extractor vessel, the desired compound may be a terpene, tetrahydrocannabinol (THC) or carbenoxolone (CBx), and the CO₂ may be circulated as a supercritical CO₂. The invention also provides a system for extracting a desired compound from a plant or a plant based material featuring a) an extractor vessel; b) a separator vessel; c) a liquid accumulator; d) at least one hot water valve; e) at least one cold water valve; f) a gas pump; g) a liquid pump; h) at least one two-way valve; i) a three-way valve; and j) a CO₂ storage vessel.

### FIELD OF INVENTION

This invention relates to methods and systems for extracting compounds from plant or plant based materials, such as terpenes, tetrahydrocannabinol (THC) and carbenoxolone (CBx).

### BACKGROUND OF INVENTION

It would be desirable to find safe, efficient and rapid ways of obtaining desired compounds such as terpenes, tetrahydrocannabinol (THC) and carbenoxolone (CBx) from plants and plant based materials. It would be especially desirable to provide a carbon dioxide (CO₂) circulation loop from which compounds could be extracted from plant based materials. Especially, it would be desirable to extract THC, terpenes, and CBx. It would be useful to extract these three distinct compounds by modulating operating temperatures and pressures within a closed circulation CO₂ loop, especially if the temperatures and pressures are modulated with an automated control system.

All publications, patent applications, patents and other reference material mentioned are incorporated by reference in their entirety. In addition, the materials, methods and examples are only illustrative and are not intended to be limiting. The citation of references herein is not to be construed as an admission that the references are prior art to the present invention.

### SUMMARY OF INVENTION

In a first aspect, the invention provides a method for extracting a desired compound such as a terpene, THC it CBx from plants and plant based materials. The method features
a) providing a plant or plant based material;
b) pumping CO₂ gas from a CO₂ storage vessel into an apparatus containing a CO₂ circulation loop;
c) circulating CO₂ through the plant or plant based material in the circulation loop;
d) extracting the desired compound; and
e) discharging the CO₂ gas from the CO₂ circulation loop.

The b) pumping CO₂ gas from a CO₂ storage vessel into an apparatus containing a CO₂ circulation loop may be performed, for instance, by opening two say valves between the CO₂ storage vessels and the CO₂ circulation loop. Further, a gas pump and a liquid pump may operate to introduce CO₂ into the CO₂ circulation loop. Still further, one or more hot water valves may be provided to heat the CO₂ once it is introduced into the CO₂ circulation loop.

The CO₂ circulation loop may feature a separator vessel and an extractor vessel. In some instances, when pressure rises above a process set point, a valve between the separator and the extractor may be closed to isolate the extractor. A gas pump and a liquid pump may operate to build pressure in the extractor. Once the pressure in the extractor rises above a target pressure, a valve between the extractor and separator may open so that the system may confirm that the separator pressure is above a set point. Then, the system may transition into circulation mode.

The c) circulating the CO₂ through the plant or plant based material in the circulation loop may last for a predetermined amount of time that may be set by a control system. The circulating may be initiated by operating a liquid pump to circulate the CO₂ and maintain pressure in the extractor. A valve between a liquid accumulator and the liquid pump may be opened. Further, a valve between the extractor and hot water valve may modulate to maintain pressure in the separator. Another valve such as a three way valve may then direct CO₂ from the separator into the liquid accumulator. During circulation a hot water valve may modulate temperature and pressure in the separator at a specific set point, and a hot water valve may modulate or maintain temperature and pressure in the extractor vessel at a specific setpoint. When extracting THC, the temperature may be maintained at, for instance, about 110° F. to 117° F., and the pressure may be maintained at, for instance, about 1200 psi to 1500 psi. When extracting CBx, the temperature may be maintained at, for instance, about 110° F. to 117° F., and the pressure may be maintained at, for instance, about 800 psi to 1050 psi. When extracting a terpene, the temperature may be maintained at, for instance, about 110° F. to 117° F., and the pressure may be maintained at, for instance, about 400 psi to 1375 psi.

The CO₂ may be circulated as a supercritical CO₂, a fluid state of CO₂ where it is held at or above its critical temperature and pressure. The supercritical CO₂ may serve as a solvent that dissolves the desired compounds, for instance, the THC, terpenes, and CBx, into a solution that can be captured and separated from the circulation loop. The supercritical CO₂ may exist at a pressure above about 100 bar and a temperature above about 300° K, for instance.

The e) discharging the CO₂ gas from the CO₂ circulation loop may be initiated when the predetermined circulation time is completed. The discharging may be a two-step process. First, a valve such as a three way valve may position to route CO₂ flow from the separator vessel to the gas pump. A valve located between the liquid accumulator and the gas pump may open to push gas into the liquid accumulator. This may continue until the pressure in the liquid accumulator rises above the liquid accumulator discharge pressure set point. Next, a valve located between the liquid accumulator and the gas pump may close. One or more valves between the process loop and CO₂ storage vessels may open, creating a flow path from the process loop to the storage vessels. The gas pump may continue to run until the pressure in the extractor vessel is less than the extractor discharge pressure set point.

At the time of a system discharge, all the gas in the system is hot and therefore expands and occupies a larger volume. Because of this, the CO₂ storage vessels may not be able to store all the circulating gas. As such, the gas may be captured and stored in the liquid accumulator vessel during the discharging.

The method may further feature f) adding gas to the system that was lost during plant or plant material change out. The method may further feature g) measuring the separator vessel pressure by a pressure transducer in the separator and adjusting optionally adjusting gas flow in response. The method may further feature h) measuring the separator vessel temperature by a thermocouple in the separator outlet line and optionally adjusting temperature in response. The method may further feature i) measuring the extractor vessel temperature by a thermocouple in the extractor outlet line and optionally adjusting temperature in response. When extracting THC, the temperature may be maintained at, for instance, about 110° F. to 117° F., and the pressure may be maintained at, for instance, about 1200 psi to 1500 psi. When extracting CBx, the temperature may be maintained at, for instance, about 110° F. to 117° F., and the pressure may be maintained at, for instance, about 800 psi to 1050 psi. When extracting a terpene, the temperature may be maintained at, for instance, about 110° F. to 117° F., and the pressure may be maintained at, for instance, about 400 psi to 1375 psi.

The method may further feature j) correcting high pressure. For instance, in the event that high pressure develops in the system during circulation, an automatic correction routine (HPC) may triggered to remove some gas from the system. This function may be triggered when the pressure in the separator vessel rises above a target set point or the pressure in the extractor vessel rises above a target set point. The j) correcting high pressure may operate for a minimum period of time and may terminate when the pressure in either of the vessels falls below the set points.

The method may further feature k) correcting low pressure. For instance, in the event the pressure in the extractor vessel falls below a set point, valves may open to provide a flow path from the CO₂ storage vessels into the process loop. The gas pump may operate to move gas into the process loop.

The method may further feature l) separating a terpene by providing a low pressure separation process. A terpene compound extraction process may require an additional process to occur after c) circulating CO₂ through the plant or plant based material in the circulation loop. Upon completion of the standard circulation mode, a valve may direct CO₂ into the gas pump located between the separator and the liquid accumulator. Valves located between the liquid accumulator and the gas pump and liquid pump may open, and the liquid pump may operate. A valve located between the extractor vessel and separator vessel may modulate the pressure in the separator vessel. The Low Pressure Separation may run for a predetermined amount of time set by the control system.

In a second aspect, the invention provides a system for extracting desired compounds such as terpenes, THC and CBx from plants and plant based materials comprising:
a) an extractor vessel;
b) a separator vessel;
c) a liquid accumulator;
d) at least one hot water valve;
e) at least one cold water valve;
f) a gas pump;
g) a liquid pump;
h) at least one two-way valve;
i) a three-way valve; and
j) a CO₂ storage vessel.

The system may further comprise k) a housing for the elements and l) an automated control system for monitoring and controlling temperature and pressure within one or more vessels. In some instances, there may be 4, 5, 6, or 7 or so two-way valves, and in some instances there may be 2, 3, 4 or so CO₂ storage vessels. There may be 1 or 2 CO₂ storage vessels that hold CO₂ gas when the system is not operating. A third CO₂ storage vessel, a makeup tank, may be used to add gas into the system and maintain operating temperatures and pressures. The system may function as a substantially closed circulation carbon dioxide loop.

The system may further feature m) a separator pressure control loop comprising a pressure transducer. Also, the system may further feature n) an extractor vessel pressure control loop comprising a pressure transducer. Still further, the system may feature o) a separator vessel temperature control loop comprising a thermocouple in the gas line on the outlet of the separator vessel. Moreover, the system may feature p) an extractor vessel temperature control loop comprising a thermocouple in the gas line on the outlet of the extractor vessel.

### BRIEF DESCRIPTION OF THE DRAWINGS

FIG. 1 demonstrates graphically the interaction of pressure and temperature for supercritical CO₂ in liquid state.

FIG. 2 provides a diagram of the elements of the system described herein. The system is a closed circulation carbon dioxide loop. It consists of an A) extractor; B) separator; C) liquid accumulator; two (2) D) hot water valves; one (1) E) cold water valve; one (1) F) gas pump; one (1) G) liquid pump; seven (7) H) two-way valves; one (1) I) three-way valve; and three (3) J) CO₂ cylinders.

FIGS. 3A and 3B depicts one preferred system described herein. The system is a closed circulation carbon dioxide loop. It consists of an A) extractor; B) separator; C) liquid accumulator; two (2) D) hot water valves; one (1) E) cold water valve; one (1) F) gas pump; one (1) G) liquid pump; seven (7) H) two-way valves; one (1) I) three-way valve; and three (3) J) CO₂ cylinders.

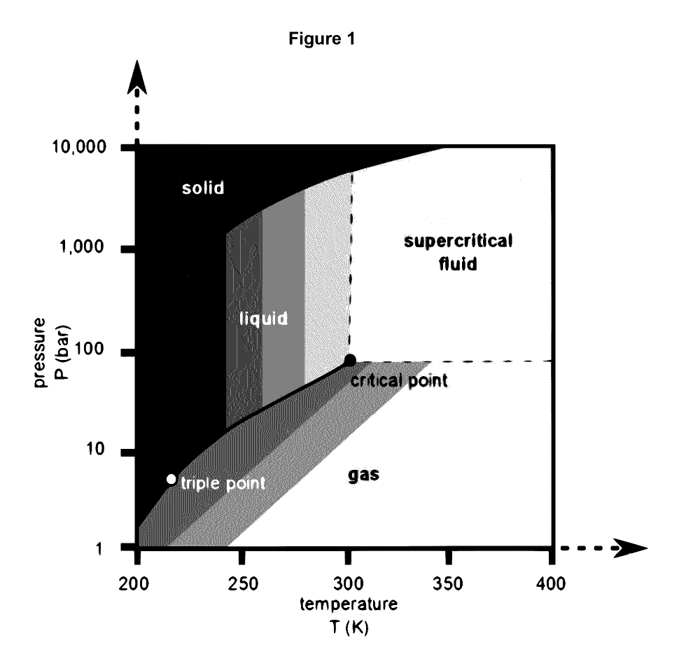

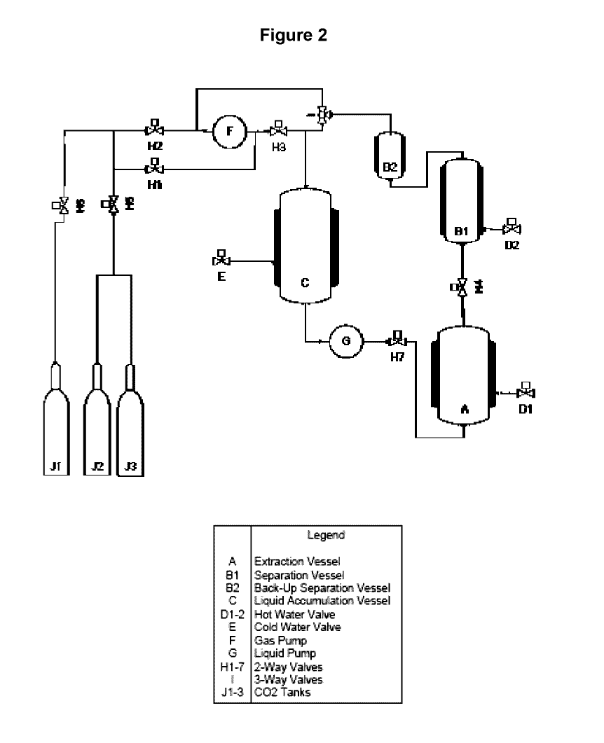

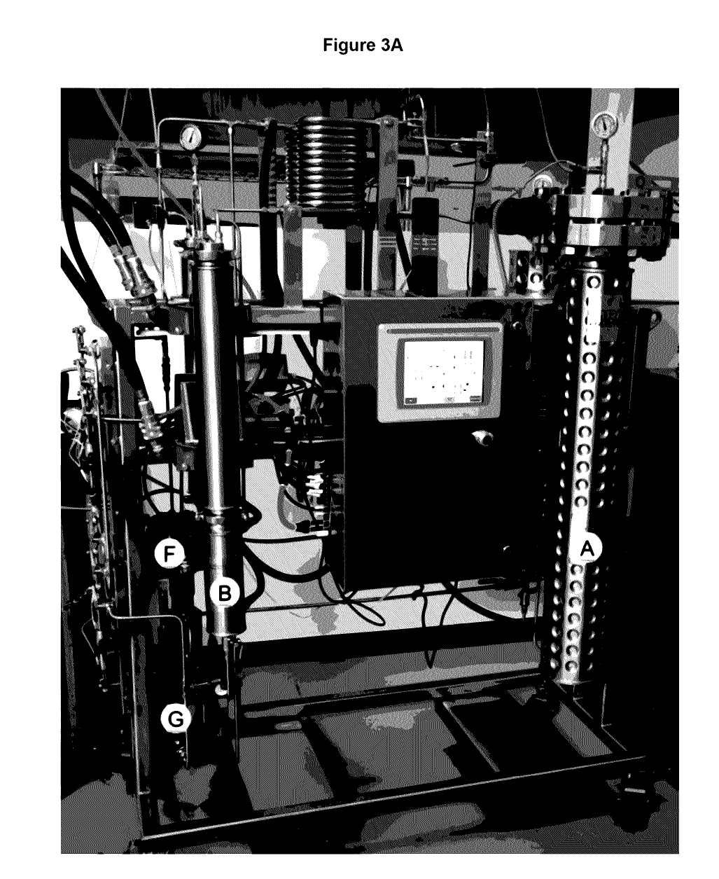

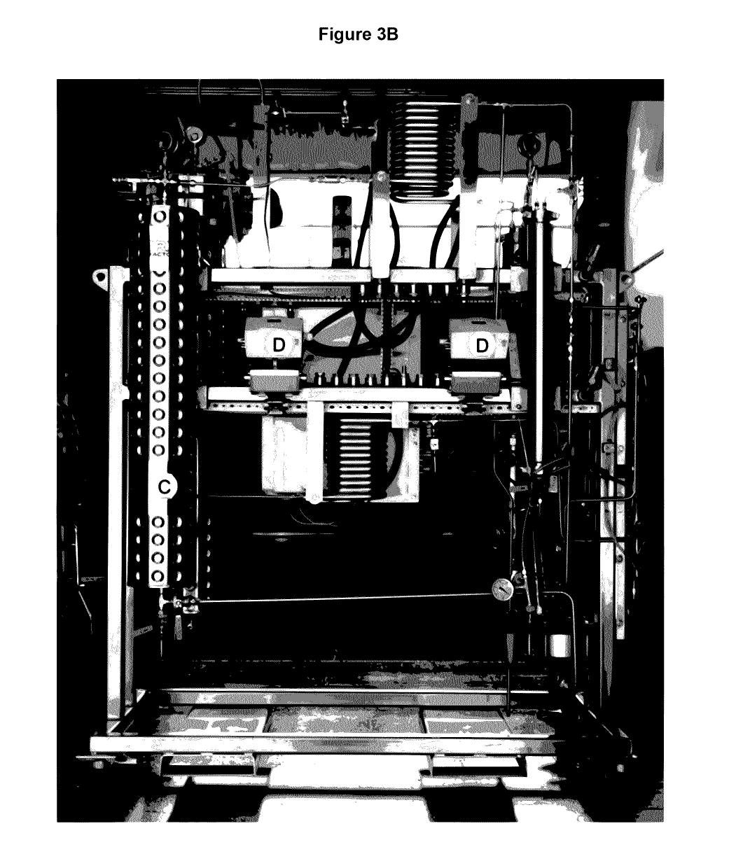

### DETAILED DESCRIPTION OF THE PREFERRED EMBODIMENTS

The present methods and systems feature the following elements and steps. The system is a closed circulation carbon dioxide (CO₂) loop, plant based material, compound extractor. The system is capable of extracting three distinct compounds from plant based material when run: 1) THC; 2) terpenes; and 3) CBx. The extraction of these three distinct compounds is achieved by modulating operating temperatures and pressures within the closed circulation CO₂ loop of the system to predetermined, precise values with an automated control system. When extracting THC, the temperature may be maintained at, for instance, about 110° F. to 117° F., and the pressure may be maintained at, for instance, about 1200 psi to 1500 psi. When extracting CBx, the temperature may be maintained at, for instance, about 110° F. to 117° F., and the pressure may be maintained at, for instance, about 800 psi to 1050 psi. When extracting a terpene, the temperature may be maintained at, for instance, about 110° F. to 117° F., and the pressure may be maintained at, for instance, about 400 psi to 1375 psi.

The automated control system contains several automatic correction functions that maintain the system at the desired temperature and pressure throughout the system extraction process.

Supercritical CO₂ is a fluid state of CO₂ where it is held at or above its critical temperature and pressure. These desired temperature and pressure ranges are created within the closed circulation loop of the system, allowing CO₂ to serve as a solvent that dissolves the chemically different 1) THC; 2) terpenes; and 3) CBx into a solution that can be captured and separated from the circulation loop. The solubility of extracted 1) THC; 2) terpenes; and 3) CBx in CO₂ vary with pressure, permitting selective extractions of each compound through temperature and pressure modulation. The relatively low temperature of the process and the stability of CO₂ allow compounds to be extracted with little damage or denaturing, and the process is non-toxic and non-flammable.

The system is a closed circulation carbon dioxide loop. It consists of an A) extractor; B) separator; C) liquid accumulator; two (2) D) hot water valves; one (1) E) cold water valve; one (1) F) gas pump; one (1) G) liquid pump; seven (7) H) two-way valves; one (1) I) three-way valve; and three (3) J) CO₂ cylinders.

There are two CO₂ storage cylinders that hold CO₂ gas when the process is not operating. A third CO₂ cylinder, the makeup tank, is used to add gas into the system and maintain operating temperatures and pressures.

There are distinct processes for each of the three compounds that the machine extracts. Each of these extraction processes utilizes the system charge, circulation and discharge steps of the process, however, the particular operating temperatures and pressure are different for each extraction process. Further, the 2) terpenes process requires an additional process step for Low Pressure Separation. When extracting THC, the temperature may be maintained at, for instance, about 110° F. to 117° F., and the pressure may be maintained at, for instance, about 1200 psi to 1500 psi. When extracting CBx, the temperature may be maintained at, for instance, about 110° F. to 117° F., and the pressure may be maintained at, for instance, about 800 psi to 1050 psi. When extracting a terpene, the temperature may be maintained at, for instance, about 110° F. to 117° F., and the pressure may be maintained at, for instance, about 400 psi to 1375 psi.

The system pre-charge step is an optional, manually initiated function that pre-fills the system with makeup gas prior to initiating a standard system charge. This function is generally used to add gas to the system that was lost during plant material change out. Upon initiation of the system pre-charge, three of the H) two-way valves open and the F) gas pump turns on. This pushes gas from the makeup tank J) CO₂ cylinders to the C) liquid accumulator vessel. When the pressure in the C) liquid accumulator rises above the pre-charge set point all valves are closed and the F) gas pump turns off, completing the pre-charge cycle.

The system charge pumps CO₂ gas from the J) CO₂ storage cylinders into the process. This function prepares the process for circulation. The system charge is initiated by opening five (5) H) two-way valves between the J) CO₂ cylinders and the process. Once the H) two-way valves are confirmed open, the F) gas pump and G) liquid pump are turned on, pushing CO₂ into the system. Two (2) D) hot water valves are opened to begin heating the CO₂ now contained in the system. When the B) separator vessel pressure raises above the process set point a H) two-way valve between the A) extractor and B) separator is closed to isolate the A) extractor. The F) gas pump and G) liquid pump continue to run to build pressure in the A) extractor. Once the pressure in the A) extractor rises above the target pressure, the H) two-way valve between the A) extractor and B) separator is opened, the system confirms that the B) separator pressure is still above the set point, and the system transitions into circulation mode.

Circulation is the steady state operation where CO₂ is circulated through the plant based material to extract the desired compounds. Circulation mode lasts for a predetermined amount of time that is set by the control system. Once the system charge is completed, circulation mode is initiated by running the G) liquid pump to circulate the CO₂ and maintain pressure in the A) extractor. One H) two-way valve between the C) liquid accumulator and the G) liquid pump is opened and one H) two-way valve between the A) extractor and D) hot water valve modulates to maintain pressure in the B) separator. The I) three-way valve then directs CO₂ from the A) separator into the C) liquid accumulator. Five of the H) two-way valves are then closed and the F) gas pump is turned off. During circulation one D) hot water valve modulates to maintain temperature and pressure in the B) separator at a specific setpoint, and one D) hot water valve modulates to maintain temperature and pressure in the A) extractor at a specific setpoint.

The discharge sequence is initiated when the predetermined circulation time is completed. The discharge sequence is a two-step process. First, the I) three-way valve positions to route CO₂ flow from the B) separator to the F) gas pump. The H) two-way valve located between the C) liquid accumulator and the F) gas pump opens to push gas into the C) liquid accumulator. This step continues until the pressure in the C) liquid accumulator rises above the liquid accumulator discharge pressure set point. Next, the H) two-way valve located between the C) liquid accumulator and the F) gas pump closes, H) two-way valves between the process and the J) CO₂ cylinders open, creating a flow path from the process to the storage tanks. The F) gas pump continues to run until the pressure in the B) extractor vessel is less than the extractor discharge pressure set point.

At the time of a system discharge, all the gas in the system is hot and therefore expands and occupies a larger volume. Because of this, the J) CO₂ cylinders cannot store all the circulating gas. This is why gas is captured and stored in the C) liquid accumulator vessel during the discharge. Without this feature, some of the gas would have to be wasted.

Each of the parameters in the process is maintained at the set point using a Proportional, Integral, Derivative feedback controller. Each of the control loops is described below.

Separator Pressure Control Loop: The separator pressure is measured by a pressure transducer in the B) separator and continually compared to the set point for the given compound. The difference between the actual pressure and the set point is called the error. A H) two-way valve between the B) separator and A) extractor opens/closes in response to pressure error signal. If the actual pressure rises above the set point, the H) two-way valve closes to reduce gas flow and lower the pressure. If the actual pressure falls below the set point, the H) two-way valve opens.

Extractor Pressure Control Loop: The A) extractor pressure is measured by a pressure transducer in the A) extractor vessel and continually compared to the set point for the given compound. The difference between the actual pressure and the set point is called the error. The G) liquid pump raises or drops in response to pressure errors. If the actual pressure rises above the set point, the G) liquid pump slows down to reduce gas flow and lower the pressure. If the actual pressure falls below the set point, the G) liquid pump speeds up. The pump speed is not allowed to go below 75% of full speed. This is necessary to maintain circulation in the system.

Separator Temperature Control Loop: The B) separator temperature is measured by a thermocouple in the gas line on the outlet of the B) separator vessel. The actual temperature is continually compared to the set point to determine the error. The D) hot water valve between the A) extractor and B) separator modulates to increase or decrease the input to the system. If the temperature rises above the set point, the D) hot water valve between the A) extractor and B) separator moves in the close direction to reduce hot water flow and allow the system to cool. If the temperature falls below the set point, D) hot water valve moves in the open direction to increase hot water flow and allow the system to heat up.

Extractor Temperature Control Loop: The A) extractor temperature is measured by a thermocouple in the gas line on the outlet of the extractor vessel. The actual temperature is continually compared to the set point to determine the error. The D) hot water valve between the A) extractor and B) separator modulates to increase or decrease the input to the system. If the temperature rises above the set point, the D) hot water valve between the A) extractor and C) liquid accumulator moves in the close direction to reduce hot water flow and allow the system to cool. If the temperature falls below the set point, the D) hot water valve between the A) extractor and C) liquid accumulator moves in the open direction to increase hot water flow and allow the system to heat up.

High Pressure Correction (HPC):

In the event that high pressure develops in the system during circulation, an automatic correction routine (HPC) is triggered to remove some gas from the system. This function is triggered when the pressure in the B) separator vessel rises above the target set point or the pressure in the A) extractor vessel rises over the target set point. The correction function operates for a minimum period of time and is terminated when the pressure in either of the vessels falls below the set points. There is delay between high pressure adjustments to prevent excessively frequent corrections.

During HPC, H) two-way valves between the process and the J) CO₂ cylinders open to make a flow path from the process back to the storage tanks and the F) gas pump turns on to push the gas into the storage tank.

Low Pressure Correction (LPC):

In the event the pressure in the A) extractor vessel falls below the A) extractor pressure set point, the system will perform a low pressure correction (LPC). This is triggered when the A) extractor pressure falls below a predetermined setpoint over a given period of time. During LPC, three (3) H) two-way valves are opened to make a flow path from the J) CO₂ cylinders into the process. The F) gas pump is started to move gas from the makeup tank into the process.

Low Pressure Separation (LPS)

The 2) terpene compound extraction process requires an additional process to occur after circulation mode is completed. Upon completion of the standard circulation mode, LPS mode is initiated. When LPS is initiated the I) three-way valve directs CO₂ into the F) gas pump located between the B) separator and the C) liquid accumulator. Two (2) H) two-way valves located between the C) liquid accumulator, F) gas pump, and G) liquid pump are opened, and the G) liquid pump is turned on. One H) two-way valve located between the A) extractor and B) separator modulates position to control the pressure in the B) separator. The Low Pressure Separation runs for a predetermined amount of time set by the control system.

Each step in the process, system pre-charge, system charge, circulation, discharge, and low pressure separation (for 2) terpene) require precise pressure, temperature, and time setponits unique to each compound. The combination of these specific pressure, temperature, and time setpoints are unique, nonobvious recipes. They contain varying, highly specified pressure, temperature, and time values that are required to be maintained within the A) extractor, B) separator, and C) liquid accumulator, throughout the entire process, to successfully complete plant material compound extraction.

We claim:

1. A method for extracting a desired compound from a plant or a plant based material comprising:
a) providing a plant or plant based material;
b) pumping CO₂ gas from a CO₂ storage vessel into an apparatus containing a CO₂ circulation loop;
c) circulating CO₂ through the plant or plant based material in the circulation loop;
d) extracting the desired compound; and
e) discharging the CO₂ gas from the CO₂ circulation loop.

2. The method of claim 1 wherein the CO₂ circulation loop comprises a separator vessel and an extractor vessel.

3. The method of claim 1 wherein the desired compound is selected from the group consisting of a terpene, tetrahydrocannabinol (THC) and carbenoxolone (CBx).

4. The method of claim 1 wherein the CO₂ is circulated as a supercritical CO₂.

5. The method of claim 1 wherein the c) circulating the CO₂ through the plant or plant based material in the circulation loop lasts for a predetermined amount of time set by a control system.

6. The method of claim 1 further comprising f) adding gas to the system that was lost during plant or plant material change out.

7. The method of claim 1 further comprising g) measuring a separator pressure by a pressure transducer in the separator and adjusting optionally adjusting gas flow in response.

8. The method of claim 1 further comprising h) measuring a separator temperature by a thermocouple and optionally adjusting temperature in response.

9. The method of claim 1 further comprising i) measuring an extractor temperature by a thermocouple and optionally adjusting temperature in response.

10. The method of claim 1 further comprising j) correcting high pressure or low pressure.

11. The method of claim 1 further comprising l) separating a terpene by providing a low pressure separation circulation of CO₂ through the plant or plant based material in the circulation loop.

12. A system for extracting a desired compound from a plant or a plant based material comprising:
a) an extractor vessel;
b) a separator vessel;
c) a liquid accumulator;
d) at least one hot water valve;
e) at least one cold water valve;
f) a gas pump;
g) a liquid pump;
h) at least one two-way valve;
i) a three-way valve; and
j) a CO₂ storage vessel.

13. The system of claim 12 wherein the desired compound is selected from the group consisting of a terpene, tetrahydrocannabinol (THC) and carbenoxolone (CBx).

14. The system of claim 12 wherein CO₂ is circulated as a supercritical CO₂.

15. The system of claim 12 further comprising k) a housing.

16. The system of claim 12 wherein CO₂ is circulated as a supercritical CO₂.

17. The system of claim 12 further comprising l) a separator pressure control loop comprising a pressure transducer.

18. The system of claim 12 further comprising m) an extractor pressure control loop comprising a pressure transducer.

19. The system of claim 12 further comprising n) a separator temperature control loop comprising a thermocouple.

20. The system of claim 12 further comprising o) an extractor temperature control loop comprising a thermocouple.
